(19) United States
(12) Patent Application Publication
(10) Pub. No.: US 2012/0209326 A1
(43) Pub. Date: Aug. 16, 2012

(54) ULTRAVIOLET LIGHT PACIFIER

(75) Inventors: Linda Marie Lutes, Mustang, OK (US); Richard James McKenzie, Butler, PA (US)

(73) Assignee: KONINKLIJKE PHILIPS ELECTRONICS N.V., EINDHOVEN (NL)

(21) Appl. No.: 13/503,759

(22) PCT Filed: Oct. 5, 2010
(86) PCT No.: PCT/IB2010/054503

§ 371 Date: Apr. 24, 2012

Related U.S. Application Data
(60) Provisional application No. 61/257,868, filed on Nov. 4, 2009.

(51) Int. Cl. A61J 17/00; A61N 5/06

(52) U.S. Cl. 606/234; 607/92

(57) ABSTRACT

An ultraviolet light pacifier (10) is provided. That is, a pacifier includes an ultraviolet light source (42) and is structured to project the ultraviolet light within the mouth of an infant via the pacifier nipple (16).

The disclosed and claimed concept relates to a pacifier and, more specifically, to a pacifier structured to project an ultraviolet light.

Pneumonia accounts for about fifteen percent of all hospital associated infections and for about a quarter of all infections acquired in intensive care units and coronary care units. One significant factor in the development of hospital associated pneumonia is mechanical ventilation. Ventilator associated pneumonia (VAP) is pneumonia that develops at, or later than, forty-eight hours after the patient has been placed on mechanical ventilation. The increased risk of pneumonia for patients on mechanical ventilation is due, in part, to microorganisms that may be on ventilation devices such as, but not limited to, endotracheal tubes. Further, bacteria can multiply on the surface of such mechanical ventilation devices and may form a protective shell, i.e. a glycocalyx, that protects the bacteria from antimicrobial agents and the patient's own defenses.

VAP is the second most common hospital acquired infection among pediatric and neonatal intensive care unit patients. Infants, especially pre-term infants, are vulnerable to VAP due to their immature lungs and weak immune systems.

Ultraviolet light, especially UV-C (short wave ultraviolet light), has been proven to reduce bacteria and provide disinfection. UV-C has, for example, been used to sanitize air, water and surfaces. UV-C has also been used as a curing agent for dental epoxies. That is, UV-C has been approved for use within a human mouth.

The disclosed concept provides for an ultraviolet light pacifier. That is, a pacifier includes an ultraviolet light source and is structured to project the ultraviolet light within the mouth of an infant. Such ultraviolet light therapy may reduce the number of bacteria which contribute to VAP. As such, the instances of VAP may be reduced. It is noted that because an infant is likely to use a pacifier for an extended period of time, the ultraviolet light provided may be less intense than the ultraviolet light used with dental epoxies.

The ultraviolet light may be produced within the pacifier or be transmitted to the pacifier. As used herein, an ultraviolet light device includes an “ultraviolet light source” and an “ultraviolet light emitter.” As used herein, a “ultraviolet light source” creates an ultraviolet light. Further, as used herein, a “ultraviolet light emitter” is the surface of the ultraviolet light device from which the ultraviolet light emerges. By way of analogy, in a common light bulb the filament creates the light, and is a source, and the light emerges from the glass bulb, which is an emitter.

Preferably, the ultraviolet light pacifier utilizes an ultraviolet light emitting diode (LED). In a typical ultraviolet LED, the light is created by passing electricity from an anode to a cathode. These elements are typically disposed under a lens or case. Thus, the emitter may be the LED lens. Alternately, the ultraviolet light source, LED or otherwise, may be coupled to one or more optic fibers. The optic fibers transmit the ultraviolet light from the source to the distal ends of the fibers. That is, in this configuration, the optic fibers act as the emitter. Thus, the ultraviolet light pacifier may either incorporate ultraviolet LEDs or have ultraviolet light transmitted into the pacifier body by optic fibers.

These and other features and characteristics of the present concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural references unless the context clearly dictates otherwise.

A full understanding of the invention can be gained from the following description of the illustrated embodiments when read in conjunction with the accompanying drawings in which:

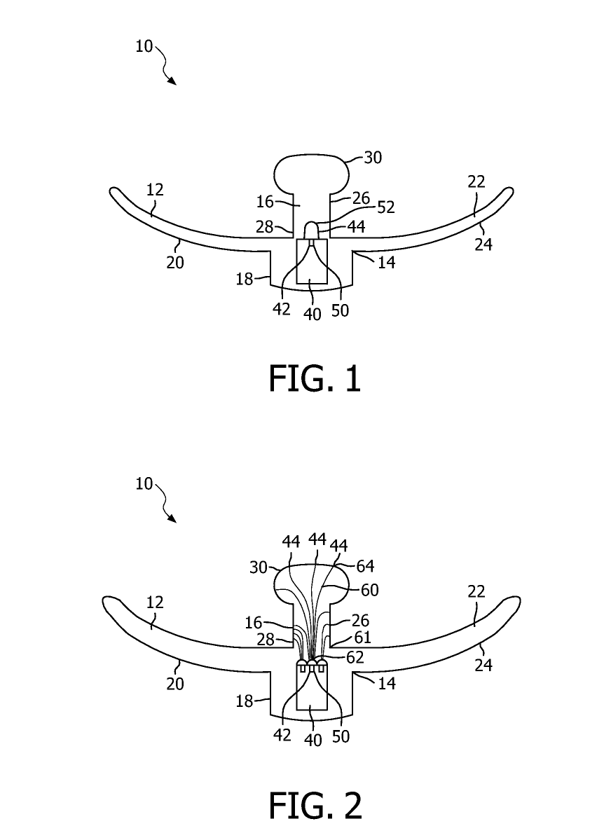

FIG. 1 is a schematic side view of a pacifier.

FIG. 2 is a schematic side view of an alternate embodiment of the pacifier having optic fibers.

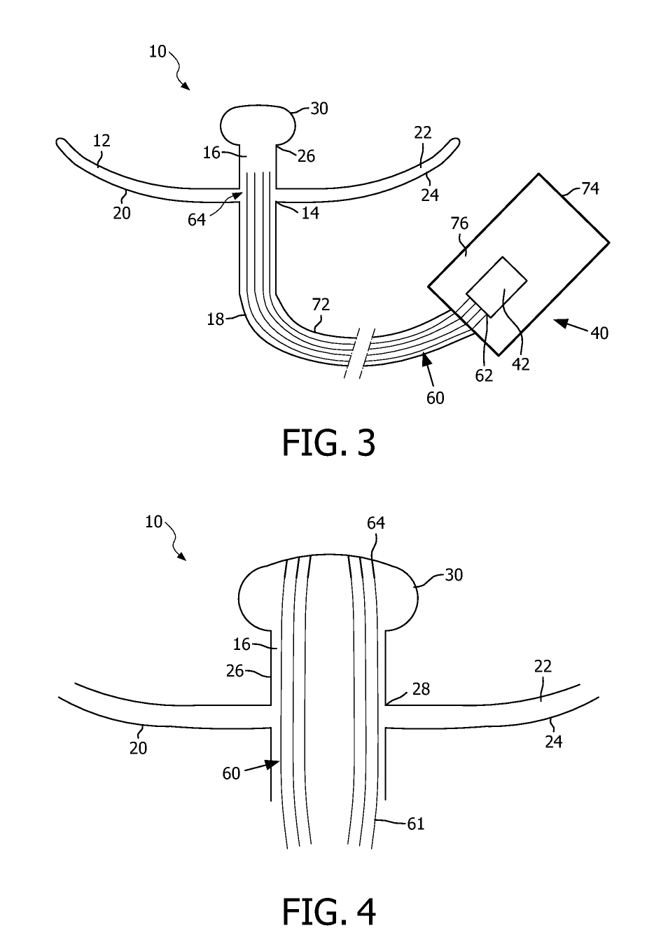

FIG. 3 is a schematic side view of an alternate embodiment of the pacifier having a base unit.

FIG. 4 is a detailed schematic side view of an alternate embodiment of the pacifier having optic fibers.

As shown in FIG. 1, an ultraviolet light pacifier 10 includes the common elements of a pacifier 12 as well as an ultraviolet light assembly 40. That is, the pacifier 12 includes a handle assembly 14 and a nipple 16. The handle assembly 14 has a handle 18 and a mouth shield 20. As is known, the mouth shield 20 is sized to prevent the infant from swallowing the pacifier. The mouth shield has an inner side 22 and an outer side 24. The nipple 16 has a, typically, elongated body 26 with a base 28 and a head 30. The nipple body 26 may be solid or hollow. The head 30, typically, has a cross-sectional area that is greater than the cross-sectional area of the base 28. The handle 18 is coupled to the mouth shield outer side 24. The nipple body base 28 is coupled to the mouth shield inner side 22

The ultraviolet light assembly 40 has at least one ultraviolet light source 42 with at least one associated ultraviolet light emitter 44. The ultraviolet light source 42 is structured to create and project ultraviolet light. The ultraviolet light emitter 44 is in light communication with the ultraviolet light source 42 and is structured to allow ultraviolet light to pass through the ultraviolet light emitter 44. The ultraviolet light emitter 44 is further structured to project ultraviolet light through the nipple 16.

In one embodiment, shown in FIG. 1, the at least one ultraviolet light source 42 is at least one ultraviolet light emitting diode 50 which has a shell or lens 52 that acts as an ultraviolet light emitter 44. The at least one ultraviolet light emitting diode 50 may be disposed within the nipple 16. Alternately, Shown in FIG. 2, the at least one ultraviolet light emitting diode 50 may be disposed within the handle 18 and structured to project ultraviolet light into the nipple 16. There may be a plurality of ultraviolet light emitting diodes 50. The nipple 16 is preferably made from a material that is substantially transparent to ultraviolet light and/or structured to refract ultraviolet light. Thus, when an infant utilizes the pacifier, ultraviolet light is projected through the nipple 16 and into the mouth.

Generally, LEDs are several millimeters in diameter. The greatest diameter is typically associated with the shell/lens 52. It is, however, foreseeable that LEDs may be made without a shell/lens 52. In this configuration, the ultraviolet light emitting diode 50 may be embedded within the material that forms the nipple 16. Thus, the nipple 16 would become the ultraviolet light emitter 44.

Another embodiment, shown in FIG. 2, provides for at least one elongated optical fiber 60, and more typically a plurality of optical fibers 60, that act as the ultraviolet light emitter 44. That is, each optical fiber 60 has an elongated body 61 with a first end 62 and a second end 64 and is structured to transmit light therethrough. Each optical fiber first end 62 is disposed in light communication with the at least one ultraviolet light source 42. Each optical fiber second end 64 is disposed in the nipple 16. Thus, ultraviolet light is transmitted from the at least one ultraviolet light source 42 through the nipple body 26 via the at least one optical fiber 60 and each optical fiber second end 64 acts as an ultraviolet light emitter 44. This allows for the ultraviolet light source 42 to be disposed in a location other than the nipple 16 whereby the nipple may be more flexible or softer.

Because the optical fibers 60 are structured to transmit light, the ultraviolet light source 42 does not have to be disposed adjacent to the nipple 16. For example, and as shown in FIG. 3, the handle assembly handle 18 may include a base unit 70 and a flexible neck 72. The base unit 70 has a housing 74 structured to enclose the ultraviolet light source 42 and which may enclose other elements typically associated with the operation of a portable light, such as, but not limited to, a control/switch, a power source or battery, and various conductors (non shown). The flexible neck 72 acts as a protective tube for the optical fibers 60. The flexible neck 72 further allows for the infant to position the pacifier as desired without having to move the base unit 70. The base unit 70 is typically larger than the pacifier handle 18 and, as such, may accommodate an ultraviolet light source 42 other than an LED. The ultraviolet light source 42 may, however, be one or more LEDs disposed in the base unit 70.

The optical fibers 60 may be configured in a variety of ways. For example, all the optical fiber second ends 64 may terminate within the nipple base 28 (FIG. 3). In this configuration, the bundle of optical fibers second ends 64 appears, and functions, similar to an LED disposed in the nipple 16. Alternatively, the optical fibers second ends 64 may be disposed throughout the nipple body 26, preferably close to, but not extending beyond, the outer surface. In such an embodiment, the longitudinal axis of each optical fiber second end 64 is preferably oriented generally normal to the surface of the nipple body 26 (FIG. 2). This has the effect of generally distributing the ultraviolet light evenly. Alternatively, noting that light emitted from an optical fiber 60 tends to project along the longitudinal axis of each optical fiber second end 64, the optical fiber second ends 64 may be oriented so that the longitudinal axis of a group of optical fiber second ends 64 point in a similar direction, as shown in FIG. 4. That is, at least one group 68 of optical fibers 60 may be associated and the longitudinal axis of each optical fiber second end 64 in the at least one group 68 is oriented generally parallel to the other optical fibers 60 in the at least one group 68. This has the effect of projecting the ultraviolet light to a selected location of the interior of the mouth. Thus, for example, the optical fiber second ends 64 may be positioned so as to project the ultraviolet light towards the back of the throat.

It is noted that, although the use of optical fibers 60 is shown in FIG. 3, as being coupled to a ultraviolet light source 42 that is remote from the nipple 16, the optical fibers 60 may also be used with the embodiment shown in FIG. 2. That is, the optical fibers 60 may be used as, for example, a device to disperse the ultraviolet light within the nipple body 26.

Although the concept has been described in detail for the purpose of illustration based on what is currently considered to be the most practical and illustrated embodiments, it is to be understood that such detail is solely for that purpose and that the concept is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An ultraviolet light pacifier comprising:
a handle assembly having a handle and a mouth shield;
said mouth shield having an inner side and an outer side;
a nipple having an elongated body with a base;
said handle coupled to said mouth shield outer side;
said nipple body base coupled to said mouth shield inner side;
a ultraviolet light assembly having at least one ultraviolet light source with at least one associated ultraviolet light emitter;
said ultraviolet light source structured to create and project ultraviolet light;
said ultraviolet light emitter in light communication with said ultraviolet light source and structured to allow ultraviolet light to pass through said ultraviolet light emitter; and
said ultraviolet light emitter structured to project ultraviolet light through said nipple.

2. The pacifier of claim 1 wherein:
said at least one ultraviolet light source is at least one ultraviolet light emitting diode; and
said at least one ultraviolet light emitting diode disposed within said nipple.

3. The pacifier of claim 2 wherein said at least one ultraviolet light emitting diode is a plurality of ultraviolet light emitting diodes.

4. The pacifier of claim 1 wherein:
said at least one ultraviolet light source is at least one ultraviolet light emitting diode;
said at least one ultraviolet light emitting diode disposed within said handle assembly; and
said at least one ultraviolet light emitting diode structured to project ultraviolet light into said nipple.

5. The pacifier of claim 4 wherein said at least one ultraviolet light emitting diode is a plurality of ultraviolet light emitting diodes.

6. The pacifier of claim 1 wherein:
said ultraviolet light emitter includes at least one elongated optical fiber;
said at least one optical fiber having a first end and a second end;
said at least one optical fiber first end in light communication with said at least one ultraviolet light source;
said at least one optical fiber second end disposed within said nipple body; and
whereby ultraviolet light is transmitted from said at least one ultraviolet light source through said nipple body via said at least one optical fiber.

7. The pacifier of claim 6 wherein said at least one elongated optical fiber is a plurality of optical fibers.

8. The pacifier of claim 7 wherein the longitudinal axis of each optical fiber second end is oriented generally normal to the surface of said nipple body.

9. The pacifier of claim 7 wherein:
within said plurality of optical fibers there is at least one group of associated optical fibers;
the longitudinal axis of each optical fiber second end in said at least one group is oriented generally parallel to the other optical fibers in said at least one group; and
whereby the ultraviolet light transmitted from said ultraviolet light source may be directed to a selected location.

10. The pacifier of claim 7 wherein each optical fiber second end terminates within the nipple base.

11. The pacifier of claim 4 wherein:
said handle assembly handle includes a base unit and a flexible neck;
said neck coupled to said base unit;
said base unit having a housing structured to enclose said ultraviolet light source;
said neck includes at least one elongated optical fiber;
said at least one optical fiber having a first end and a second end;
said at least one optical fiber first end in light communication with said at least one ultraviolet light source;
said at least one optical fiber second end disposed within said nipple body; and
whereby ultraviolet light is transmitted from said ultraviolet light source through said nipple body via said at least one optical fiber.

12. The pacifier of claim 11 wherein said at least one elongated optical fiber is a plurality of optical fibers.

13. The pacifier of claim 12 wherein the longitudinal axis of each optical fiber second end is oriented generally normal to the surface of said nipple body.

14. The pacifier of claim 12 wherein:
within said plurality of optical fibers there is at least one group of associated optical fibers;
the longitudinal axis of each optical fiber second end in said at least one group is oriented generally parallel to the other optical fibers in said at least one group; and
whereby the ultraviolet light transmitted from said ultraviolet light source may be directed to a selected location.

15. The pacifier of claim 12 wherein each optical fiber second end terminates within the nipple base.

16. The pacifier of claim 11 wherein said ultraviolet light source is a plurality of ultraviolet light emitting diodes.

17. The pacifier of claim 1 wherein said nipple body is made from a material structured to refract ultraviolet light.

18. The pacifier of claim 1 wherein said nipple body is made from a material substantially transparent to ultraviolet light.
